(19) United States
(12) Patent Application Publication
(10) Pub. No.: US 2026/0250669 A1
(43) Pub. Date: Aug. 27, 2026

(54) sgRNA Highly Targeting Human HLA-A Gene, and Composition and Application Thereof

(71) Applicants: Tianhai Yuanqi Biotechnology (Tianjin) Co., Ltd., Tianjin City (CN); Hematology Hospital, Chinese Academy of Medical Sciences (Institute of Hematology, Chinese Academy o, Tianjin (CN); Hebei Xiong'an Yuanqi Biotechnology Co., Ltd., Hebei Province (CN)

(72) Inventors: Tao CHENG, Tianjin (CN); Shuquan RAO, Tianjin (CN); Linping HU, Tianjin (CN); Sha HAO, Tianjin (CN); Yiwen HAN, Tianjin (CN)

(21) Appl. No.: 19/234,329

(22) Filed: Jun. 11, 2025

(30) Foreign Application Priority Data
Jun. 17, 2024 (CN) 202410772696.3

(51) Int. Cl. C12N 15/11; C12N 9/22; C12N 15/90

(52) U.S. Cl. CPC C12N 15/11; C12N 9/226; C12N 15/907; C12N 2310/20

(57) ABSTRACT

The present invention provides an sgRNA. The nucleotide sequence of the sgRNA is shown in one of SEQ ID Nos. 1-21. The sgRNA provided by the present disclosure can knock out or knock down a human HLA-A gene efficiently, and almost entirely covers the sgRNA of the HLA-A genotype of Chinese population. The sgRNA provided by the present disclosure has high targeting, no obvious influence on the expression of HLA-B, HLA-C and HLA-II molecules after knocking out or knocking down an HLA-A gene in a human hematopoietic cell and low cytotoxicity, shows obvious advantages in the aspect of gene therapy, and has great clinical application prospect in the field of cell therapy.

### CROSS REFERENCE

This application claims priority to Chinese Patent Application No. 202410772696.3, first filed with the China National Intellectual Property Administration (CNIPA) on Jun. 17, 2024 and entitled “sgRNA HIGHLY TARGETING HUMAN HLA-A GENE, AND COMPOSITION AND APPLICATION THEREOF”, which is hereby incorporated herein by reference in its entirety.

### INCORPORATION BY REFERENCE

This application contains a sequence listing XML, which has been submitted electronically and is hereby incorporated by reference in its entirety. Said Sequence Listing XML, created on Jun. 4, 2025, is named “20250528_NZ240030_PO_US_filing_SEQ_LIST.xml”, and is 19,000 bytes in size.

### TECHNICAL FIELD

The present disclosure relates to the field of biotechnology, and in particular, to an sgRNA highly targeting a human HLA-A gene, and a composition and application thereof.

### BACKGROUND

Immune rejection response is mainly mediated by recipient immune cells recognizing major histocompatibility complex (MHC) molecules on the surface of non-self donor cells. Human MHC molecules are encoded by human leukocyte antigen (HLA) genes, and HLA-A, B, C and DR encoded by classical HLA molecules are the main molecular targets of allograft rejection. The immune rejection response is an important reason of the failure in many treatment means. For example, hematopoietic stem cell (HSC) transplantation is an important means for the treatment of various malignant hematological diseases and immune dysfunction, and the success of HSC transplantation first needs to overcome the immune barrier (immunological rejection) of the transplantation.

Knocking down or knocking out HLA-A can reduce transplantation rejection, thereby significantly improving the success rate of HLA matching and expanding the application of clinical allograft. It has been studied that the HLA-A gene of hematopoietic cells derived from umbilical cord blood was knocked out by a zinc finger nuclease (ZFN) method, which increased the matching rate of allogeneic transplantation and maintained the ability of the hematopoietic cells on in vivo implantation and lineage differentiation. However, there are many defects and deficiencies in the method of editing the HLA-A gene by ZFN. For example, 1) the efficiency of knocking out the HLA-A gene by the ZFN technology is low and only about 10%, and the clinical application prospect is poor. 2) ZFN gene editing is easy to miss the target and produces high cytotoxicity. The cleavage of DNA by ZFN requires dimerization of two Fok I cleavage regions, and requires at least one recognition unit to bind DNA. Although the DNA recognition domain has strong specific recognition ability, the cleavage process of ZFN does not depend entirely on the formation of a homodimer, so once a heterodimer is formed, it is likely to cause off-target effect, which may eventually lead to DNA mismatch and sequence change, resulting in strong cytotoxicity. When these adverse effects accumulate too much, beyond the scope of cell repair mechanism, cell apoptosis will be caused. 3) The ZFN method is limited by the existing research methods in the field of biology, and the accuracy and consequences of the operation inside the cell are difficult to predict. If ZFN causes related gene mutation, it may lead to a series of unexpected consequences, and even cause cancer in the application field related to the human body. Up to now, the ZFN technology can only be used for in vitro operation. After the cells extracted from the human body are treated, the cells are introduced and reinfused into patients. However, introducing related ZFN components directly into patients for gene editing has great potential risks and low efficiency. The above limitations make ZFN operation more complicated and difficult to popularize and apply. Therefore, at present, there is no effective means to knock down or knock out the HLA-A gene such as human hematopoietic cells, and it is urgent to solve the above problems.

### SUMMARY

### Technical Problem to be Solved

One aspect of the present invention is to provide an sgRNA highly targeting a human HLA-A gene, and a composition and application thereof, in view of the problem of lack of an effective means to efficiently knock down and/or knock out a human HLA-A gene in the prior art.

Specifically, the inventor creatively designed a small guide RNA (sgRNA) based on a CRISPR gene editing system, which can knock down and/or knock out the human HLA-A gene efficiently after being applied to the CRISPR gene editing system alone or in combination, thereby solving the above problems in the prior art.

### Technical Solution

an sgRNA, where the nucleotide sequence of the sgRNA is shown in one of SEQ ID Nos. 1-21.

In the embodiments of the present disclosure, by the CRISPR gene editing system, the sgRNA is used to recognize and knock down and/or knock out the human HLA-A gene in a cell, the efficiency of the sgRNA in a 293T cell line can reach 20% or above, and the highest efficiency on hematopoietic cells derived from umbilical cord blood can reach 97%, which is far superior to the method in the prior art. The efficiencies of three sgRNAs such as HLA-A-ex2-g13, HLA-A-ex3-g167 and HLA-A-ex2-g452 are the best. Therefore, in some embodiments of the present disclosure, the nucleotide sequence of the sgRNA is shown in SEQ ID No. 1, SEQ ID No. 2 or SEQ ID NO. 3.

In some embodiments of the present disclosure, the nucleotide sequence shown in SEQ ID Nos. 1-21 can be reasonably modified. The modification includes, but is not limited to, substitution, insertion, deletion, exchange or replacement regions of one or more nucleotides for improvement, and modifications on the nucleotide sequence by other groups.

Another aspect of the present disclosure is to provide a composition of sgRNA. The composition includes one or more sgRNAs selected from the nucleotide sequences shown in SEQ ID Nos. 1-21.

Preferably, in some embodiments of the present disclosure, the composition includes one or more sgRNAs selected from the nucleotide sequences shown in SEQ ID No. 1, SEQ ID No. 2 or SEQ ID No. 3.

In some embodiments of the present disclosure, the nucleotide sequence in the composition can be reasonably modified. The modification includes, but is not limited to, substitution, insertion, deletion, exchange or replacement regions of one or more nucleotides for improvement, and modifications on the nucleotide sequence by other groups.

Another aspect of the present disclosure is to provide a use of the sgRNA or the composition to preparation of a product for knocking out or knocking down a human HLA-A gene. The human HLA-A gene can be knocked out or knocked down in vivo or in an in vitro culture environment.

Another aspect of the present disclosure is to provide a use of the sgRNA or the composition to preparation of a medicine for preventing or reducing immune rejection response.

Another aspect of the present disclosure is to provide a recombinant vector. The recombinant vector includes the above nucleotide sequence. In some other embodiments of the present disclosure, the recombinant vector can also load the nucleotide sequence encoding Cas protein at the same time.

Another aspect of the present disclosure is to provide a kit. The kit includes the sgRNA, the composition or the recombinant vector. In some other embodiments of the present disclosure, the kit further includes Cas protein or an expression vector of Cas protein.

Another aspect of the present disclosure is to provide a drug composition. The drug composition includes the sgRNA, the composition or the recombinant vector, and a pharmaceutically acceptable vector.

Another aspect of the present disclosure is to provide a method for improving the efficiency of knocking out or knocking down a human HLA-A gene in a cell, including:
step 1): transferring the sgRNA, the composition or the recombinant vector into the cell;
step 2): transferring Cas protein or an expression vector of Cas protein into the cell; and
step 3) growing the cell.

In some embodiments of the present disclosure, the cell can grow in vivo or in an in vitro culture environment.

### Beneficial Effects

The sgRNA provided by the present disclosure can knock out or knock down a human HLA-A gene efficiently, and almost entirely covers the sgRNA of the HLA-A genotype of Chinese population. The sgRNA provided by the present disclosure has high targeting, no obvious influence on the expression of HLA-B, HLA-C and HLA-II molecules after knocking out or knocking down an HLA-A gene in a human hematopoietic cell and low cytotoxicity, shows obvious advantages in the aspect of gene therapy, and has great clinical application prospect in the field of cell therapy.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1 is a result diagram of flow detection of HLA-A expression in a 293T tool cell according to an embodiment of the present disclosure, where the result shows high expression of HLA-A protein on the surface of the 293T cell, which is significantly different from a negative group, and the result indicates that the 293T cell can be used as a tool cell for HLA-A sgRNA library screening;

FIG. 2 is a flow result diagram of a virus infection group after Puro drug screening according to an embodiment of the present disclosure, where in the figure, cells with HLA-A low expression are clustered, the HLA-A expression of the cells is entirely shifted leftwards, the proportion of the HLA-A low expression population is increased, and through flow sorting, the cells with HLA-A low expression reach 10%, the cells with middle expression reach 70% and the cells with high expression reach 10%;

FIG. 3 is a result diagram of analyzing the enrichment situation of an HLA-A low expression group compared with an unsorted group sgRNA according to an embodiment of the present disclosure, so that 24 effective sgRNAs are screened out;

FIG. 4 is a result diagram of the knock-out effect of 21 sgRNAs detected and screened by a transient transfection method on a 293T cell line according to an embodiment of the present disclosure, where the figure shows the proportion of the cells with HLA-A low expression after knock-out of each sgRNA, the proportion of the HLA-A knock-out cells in the HLA-A-ex2-g13 group with the best knock-out effect can reach 60%, and in addition, the HLA-A gene site targeted by each sgRNA is marked in the figure, P is Promoter, Ex is exon, and Int is Intron;

FIG. 5 is a result diagram of knock-out sites HLA-A-ex2-g13 (SEQ ID No. 1), HLA-A-ex3-g167 (SEQ ID No. 2) and HLA-A-ex2-g452 (SEQ ID No. 3) on a human HLA-A gene according to an embodiment of the present disclosure;

FIG. 6 is a result diagram of further detecting the knock-out effect of three sgRNAs: HLA-A-ex2-g13 (SEQ ID No. 1), HLA-A-ex3-g167 (SEQ ID No. 2) and HLA-A-ex2-g452 (SEQ ID No. 3) with the best HLA-A protein knock-out effect on a 293T cell line on hematopoietic cells derived from umbilical cord blood according to an embodiment of the present disclosure, where the flow diagram shows a gating strategy of CD34⁺, CD34⁺CD90⁺ and CD34⁺CD90⁻ cells;

FIG. 7 and FIG. 8 are result diagrams of the knock-out effect of HLA-A-ex2-g13 (SEQ ID No. 1), HLA-A-ex3-g167 (SEQ ID No. 2) and HLA-A-ex2-g452 (SEQ ID No. 3) on hematopoietic cells derived from umbilical cord blood according to an embodiment of the present disclosure, where FIG. 7 shows that three sgRNAs can effectively knock out HLA-A protein on the surfaces of CD34⁺, CD34⁺CD90⁺ and CD34⁺CD90⁻ cells, the proportions of the CD34⁺CD90⁺ cells knocked out by the three sgRNAs are 94% or above, the proportion of the HLA-A knock-out cells on CD34⁺CD90⁺ in the HLA-A-ex2-g13 (SEQ ID No. 1) group can reach 97%, and FIG. 8 shows that the proportion of the HLA-A positive cell population after knock-out of the three sgRNAs is significantly reduced through statistical analysis;

FIG. 9 and FIG. 10 are specificity result diagrams according to an embodiment of the present disclosure, where FIG. 9 shows that compared with a control group, there is no significant difference in the HLA-A on the surfaces of the CD34⁺CD90⁺ cells after HLA-A is knocked out by three sgRNAs, indicating that the three sgRNAs have significant specificity for knocking out HLA-A in a targeted manner, the expression of HLA-B and HLA-C molecules on the surfaces of CD34⁺, CD34⁺CD90⁺ and CD34⁺CD90⁻ cells are not affected after HLA-A is knocked out by the three sgRNAs, and FIG. 10 shows that the proportion of the HLA-ABC positive cell population does not change significantly after knock-out of the three sgRNAs through statistical analysis; and

FIG. 11 and FIG. 12 are specificity result diagrams according to an embodiment of the present disclosure, where FIG. 11 shows that compared with a control group, there is no significant difference in the expression of HAL-DR molecules on the surfaces of CD34+CD90+ cells after HLA-A is knocked out by three sgRNAs, and FIG. 12 shows that the proportion of the HLA-DR positive cell population does not change significantly after knock-out of the three sgRNAs through statistical analysis. This indicates that the three sgRNA have significant specificity of knocking out HLA-A in a targeted matter, and the expression of HLA-II molecules on the surfaces of CD34+, CD34+CD90+ and CD34+CD90− cells are not affected after HLA-A is knocked out by the three sgRNAs.

### SEQUENCE DESCRIPTION

Sequence Table

| Sequence Number | Sequence Name | Sequence |
|---|---|---|
| SEQ ID No. 1 | HLA-A-ex2-g13 | GGATGTGAAGAAATACCTCA |
| SEQ ID No. 2 | HLA-A-ex3-g167 | GGACCTGCGCTCTTGGACCG |
| SEQ ID No. 3 | HLA-A-ex2-g452 | GGATGGAGCCGCGGGCGCCG |
| SEQ ID No. 4 | HLA-A-ex3-g448 | GGCCGCCTCCCACTTGCGCT |
| SEQ ID No. 5 | HLA-A-ex2-g274 | CTGGTTGTAGTAGCCGCGCA |
| SEQ ID No. 6 | HLA-A-ex2-g440 | CGTGTCCCGGCCCGGCCGCG |
| SEQ ID No. 7 | HLA-A-ex4-g107 | CACAGCCGCCCACTTCTGGA |
| SEQ ID No. 8 | HLA-A-ex5-g410 | ATTGCTGGCCTGGTTCTCCT |
| SEQ ID No. 9 | HLA-A-promoter-g258 | ACCCAATGGGAGTGAGAACT |
| SEQ ID No. 10 | HLA-A-ex4-g461 | GTCCTCCCCATCCCGCTGCC |
| SEQ ID No. 11 | HLA-A-ex2-83 | GAGCCAGAGGATGGAGCCGC |
| SEQ ID No. 12 | HLA-A-promoter-190 | ACCCAGTTCTCACTCCCATT |
| SEQ ID No. 13 | HLA-A-promoter-57 | GAGAGGGAGAAAAGAAACTG |
| SEQ ID No. 14 | HLA-A-ex4-g227 | AGGTCAGTGTGATCTCCGCA |
| SEQ ID No. 15 | HLA-A-ex1-g269 | GACCCCGCACTCACCCGCCC |
| SEQ ID No. 16 | HLA-A-promoter-28 | CTGGAAACCCGACACCCAAT |
| SEQ ID No. 17 | HLA-A-ex1-g35 | GAGGGTTCGGGGCGCCATGA |
| SEQ ID No. 18 | HLA-A-promoter-g63 | TCTGGAAACCCGACACCCAA |
| SEQ ID No. 19 | HLA-A-int2-g404 | GGGGGACTGGGCTGACCGCG |
| SEQ ID No. 20 | HLA-A-ex2-g246 | CGGCTCCATCCTCTGGCTCG |
| SEQ ID No. 21 | HLA-A-promoter-g118 | GGGAGAATCTGAGTCCCGGT |

### DETAILED DESCRIPTION OF THE EMBODIMENTS

The present invention provides an sgRNA and application thereof. Those skilled in the art can learn from the contents of the specification to appropriately improve process parameters. It should be particularly noted that all similar substitutions and modifications are apparent to those skilled in the art and are considered to be included within the present invention; and related persons can obviously make a modification or a proper change and combination on the content of this paper without departing from the content, the spirit and the scope of the present invention, so as to implement and apply the technology of the present invention.

In the present invention, unless otherwise specified, scientific and technical terms used herein have the meaning as commonly understood by those skilled in the art. Unless otherwise expressly stated, throughout the specification and claims, the term “including” or its variations such as “including” or “comprising” will be understood as including the stated elements or components, but not excluding other elements or components. The terms “one” (“a”, “an” and “the”) include plural referents. The term “a plurality of” refers to two or more. The terms “such as”, “for example” and the like are intended to refer to exemplary embodiments and are not intended to limit the scope of the present disclosure.

In the present disclosure, when the value range is provided, it should be understood that, unless the context clearly indicates otherwise, the range includes endpoints, and each intermediate value between the upper and lower limits of the range and any other specified value or intermediate value within the specified range and any value within a smaller range between the specified values are covered.

In the present disclosure, the term “about” generally refers to the range of 0.5%-10% above or below the specified value, such as 0.5%, 1%, 1.5%, 2%, 2.5%, 3%, 3.5%, 4%, 4.5%, 5%, 5.5%, 6%, 6.5%, 7%, 7.5%, 8%, 8.5%, 9%, 9.5% or 10% above or below the specified value.

In the present disclosure, unless otherwise specified, scientific and technical terms used herein have the meanings commonly understood by those skilled in the art. The definitions of common terms in molecular biology can be referenced to Lewin's GENES, Twelfth Edition, Jocelyn E. Krebs, Elliott S. Goldstein, Stephen T. Kilpatrick, Press: Jones & Bartlett Learning. The definitions of common terms in biochemistry can be referenced to Lehninger Principles of Biochemistry, Eighth Edition, David L. Nelson, Michael M. Cox, Press: W. H. Freeman. The definitions of common terms in cell biology can be referenced to Molecular Biology of the Cell, Sixth Edition, Bruce Alberts, Alexander Johnson, Julian Lewis, David Morgan, Martin Raff, Keith Roberts, Peter Walter, Press: Garland Science. The definitions of common terms in genetics can be referenced to Genetics: Analysis of Genes and Genomes, Eighth Edition, Daniel L. Hartl, Maryellen Ruvolo, Press: Jones & Bartlett Learning.

Unless otherwise specified, the experimental technologies in the specification adopt the conventional technologies of immunology, biochemistry, chemistry, molecular biology, microbiology, cell biology, genomics and recombinant DNA, which can be referenced to the following standard books: Molecular Cloning: A Laboratory Manual, Cell Biology: A Laboratory Handbook, and the like.

### Definitions

The term “knock-out” in the present disclosure is used to describe the process of completely or partially inactivating a specific gene in an organism by a gene editing technology. The knock-out technology usually involves the use of a gene editing tool, such as a CRISPR-Cas9 system, to accurately position and cut off the DNA sequence of a target gene. After the DNA sequence is cut off, a cell usually tries to repair the broken DNA through a non-homologous end joining (NHEJ) or Homology directed repair (HDR) mechanism. However, these repair processes may lead to the insertion, deletion or substitution of gene sequences, thus making the target gene lose the original function or expression level.

The term “knock-down” in the present disclosure refers to reducing the expression level of a certain gene in an organism by a specific technical means, rather than completely eliminating the function of the gene, for example, implemented through antisense RNA, ribonuclease or interference (CRISPRi) of the CRISPR-Cas system. The main difference between knock-down and knock-out is the degree: knock-out refers to completely or partially eliminating the function of the gene, while knock-down refers to reducing the expression level of the gene. Therefore, knock-down allows researchers to research the influence of the reduced expression quantity of the gene is reduced, rather than the influence of the complete deletion.

The term “HLA-A” in the present disclosure is the abbreviation of human leukocyte antigen A, also known as MHC class I antigen. It is a glycoprotein expressed on the surfaces of almost all cells in the human body, belonging to a part of major histocompatibility complex (MHC). The heavy chain of the HLA-A molecule is about 45 kDa, and the gene thereof includes 8 exons. The exon 1 encodes a lead peptide, the exons 2 and 3 encode α1 and α2 domains (the two bind to peptide), the exon 4 encodes an α3 domain, the exon 5 encodes a transmembrane region, and the exons 6 and 7 encode a cytoplasmic tail. In particular, the polymorphisms in the exon 2 and the exon 3 are related to the peptide binding specificity of each HLA-A molecule, and the typing of these polymorphisms is usually used for the matching of bone marrow and kidney transplantation.

### CRISPR-Cas System:

The CRISPR-Cas system widely exists in chromosomes of bacteria and archaea, which is related to its immunity. The CRISPR-Cas system is used to defend against foreign genetic materials and gain the ability to resist phages. The CRISPR-Cas system is a third generation gene editing technology after the introduction of ZFN, TALENs and other gene editing technologies. The CRISPR-Cas system can be divided into three main types: Type I, Type II and Type III. The CRISPR Type I system is the earliest discovered and researched system, which mainly exists in most bacteria and archaea. The system includes a plurality of Cas proteins and a plurality of CRISPR RNAs (crRNA), forming a complicated Cas protein complex. The complex recognizes and shears a target fragment through the complementary pairing of crRNA and exogenous DNA. The Type I system is characterized by having a plurality of Cas proteins. The Cas protein has RNA-dependent DNA nuclease activity and is responsible for shearing the target DNA. The CRISPR Type II system, also known as a CRISPR/Cas9 system, is a gene editing tool most widely used at present. The CRISPR/Cas9 system only requires Cas9 protein and trans-activating CRISPR-derived RNA (trans-activating crRNA, tracrRNA) to form a complex, so that a target DNA fragment can be recognized and sheared. The Cas9 protein has two nuclease activities of HNH and RuvC, which can respectively shear two chains of the target DNA. The CRISPR Type III system has the structure and function similar to those of the Type I system, but has more Cas proteins and a more complicated mechanism. Similar to the Type I system, the Type III system also requires a plurality of Cas proteins and crRNAs to form a complex, and recognizes and shears a target fragment through the complementary pairing with the exogenous DNA. However, the shearing mechanism of the Type III system is different from that of the Type I system, which depends on the RNA enzyme activity of the Cas protein to cut the target DNA. In the embodiments of the present disclosure, the sgRNA is used for the Type II CRISPR-Cas system.

### Cas Protein:

In the embodiments of the present disclosure, the Cas protein used by the CRISPR-Cas system is mainly Cas9. Cas9 is an endonuclease. Under the guidance of the sgRNA(small guide RNA), Cas9 can accurately identify and cut DNA sequences. In some embodiments of the present disclosure, Cas9 protein can be isolated or loaded on a vector for intracellular expression. In addition to Cas9, in some other embodiments of the present disclosure, other accessory proteins may be included, including, but not limited to, Cas1, Cas2 and the like. The accessory proteins may play an auxiliary role in the DNA cutting process, or participate in other links of the CRISPR system, such as DNA acquisition and processing.

In the embodiments of the present disclosure, Cas protein may be modified. The modification may include mutation, insertion or deletion of amino acid residues, codon optimization, and some other chemical modifications. The objective may be to change the activity or specificity of Cas protein without changing the main function of Cas protein. For example, the US patent US20180100148A1 describes the mutation of nucleic acid and protein of Streptococcus pyogenes Cas9 to reduce the off-target effect of the system.

sgRNA and Combination Thereof:

In the embodiment of the present disclosure, the nucleotide sequence of the sgRNA shown in SEQ ID Nos. 1-21 comes from an sgRNA library built in the Institute of Hematology, Chinese Academy of Medical Sciences (unpublished). According to the HLA-A gene information (HLA-A major histocompatibility complex, class I, A [Homo sapiens (human)], Gene ID:3105) provided by NCBI, we designed a guide RNA(gRNA) database. The library includes 520 sgRNAs, of which 50 are negative controls. The inventor obtained the sgRNA and the combination thereof after library screening. After the nucleotide sequence is known, it can be obtained by suitable methods in the prior art, including but not limited to chemical synthesis, polymerase chain reaction (PCR) and the like.

In the gene editing process, the sgRNA can be used alone, and in some embodiments of the present disclosure, about 20%, about 30%, about 40%, about 50%, about 60%, about 65%, about 70%, about 75%, about 80%, about 85%, about 90%, about 95%, about 98% and about 99% of editing efficiency can be achieved; and the sgRNA can be used in combination, or other sgRNAs can be added, and in some other embodiments of the present disclosure, about 20%, about 30%, about 40%, about 50%, about 60%, about 65%, about 70%, about 75%, about 80%, about 85%, about 90%, about 95%, about 98% and about 99% of editing efficiency can also be achieved. In some embodiments of the present disclosure, the sgRNA can be loaded in the same container, or can be loaded in different containers and transferred into cells.

The nucleotide sequence of the above sgRNA can be modified. The modification includes, but is not limited to, substitution, insertion, deletion, exchange or replacement regions of one or more nucleotides for improvement, and modifications on the nucleotide sequence by other groups. In some embodiments of the present disclosure, the mutated sgRNA nucleotide sequence may have at least 20%, at least 30%, at least 40%, at least 50%, at least 60%, or at least 70%, at least 80%, at least 85%, at least about 90%, at least about 95%, at least about 96%, at least about 97%, at least about 98% or at least bout 99% of identity compared with SEQ ID No. 1-21.

### Vector or Recombinant Vector:

In the embodiments of the present disclosure, the provided vector or recombinant vector at least includes the nucleotide sequence of the sgRNA. The objective is to transfer nucleic acid to a target cell for gene editing. In some embodiments of the present disclosure, the vector or recombinant vector further includes a nucleotide sequence encoding Cas protein, which is operably connected to a suitable promoter.

In some embodiments of the present disclosure, the vector or recombinant vector can be encapsulated into virus or virus-like particles for transfer to target cells. Examples of the vector or recombinant vector include, but are not limited to, a plasmid vector, a lentivirus vector, an adenovirus vector, an adeno-associated virus vector or a retroviral vector. In some embodiments of the present disclosure, the vector may be a linear vector or a circular vector. The vector may be a non-viral vector such as plasmid, or a virus vector, or a vector using a transposon. The vector may include a regulatory sequence such as a promoter and a terminator, and a tag sequence such as a drug-resistant gene and a reporter gene.

### Applied Cells:

In the embodiments of the present disclosure, performing gene editing by the sgRNA or the composition of the sgRNA to knock out or knock down the human HLA-A gene can be applied to any cell including the human HLA-A gene. For example, in some embodiments of the present disclosure, the cells are human cells, including but not limited to, Embryonic stem cells, induced pluripotent stem cells, germ cells, fibroblasts, oligodendroglial cells, glia cells, hematopoietic stem cells/hematopoietic progenitor cells, neuron group cells, neurons, muscle cells, bone cells, liver cells, pancreatic cells, retinal cells, cancer cells, T cells, B cells, NK cells, fetal cardiomyocytes, myofibroblasts, mesenchymal stem cells, autologous transplanted expanded cardiomyocytes, adipocytes, differentiated totipotent cells, pluripotent cells, blood stem cells, myoblasts, adult stem cells, bone marrow cells, mesenchymal cells, parenchymal cells, epithelial cells, endothelial cells, mesothelial cells, fibroblasts, osteoblasts, chondrocytes, exogenous cells, endogenous cells, stem cells, hematopoietic stem cells, bone marrow-derived cells, cardiomyocytes, skeletal cells, fetal cells, undifferentiated cells, pluripotent group cells, monopotential group cells, monocytes, cardiac myoblasts, skeletal myoblasts, macrophages, capillary endothelial cells, xenogeneic cells, allogeneic cells or postpartum stem cells. In some other embodiments of the present disclosure, the cells can also be cells that grow in other organisms but include human HLA-A.

In some embodiments of the present disclosure, the gene editing may occur in vivo or in vitro. Elements of the CRISPR-Cas system described in the present disclosure can be transferred into the cells by any suitable method, for example, including but not limited to transfection, viral infection, electrotransfection RNP and the like.

To make those skilled in the art better understand the technical solutions of the present invention, the present invention will be further described in detail below in combination with the specific embodiments.

### EMBODIMENTS

The sgRNA library in this embodiment is built in the Institute of Hematology, Chinese Academy of Medical Sciences (unpublished). According to the HLA-A gene information (HLA-A major histocompatibility complex, class I, A [Homo sapiens (human)], Gene ID:3105) provided by NCBI, we designed a guide RNA(gRNA) database. The library includes 520 sgRNAs, of which 50 are negative controls.

### Embodiment 1: HLA-A sgRNA Library Screening

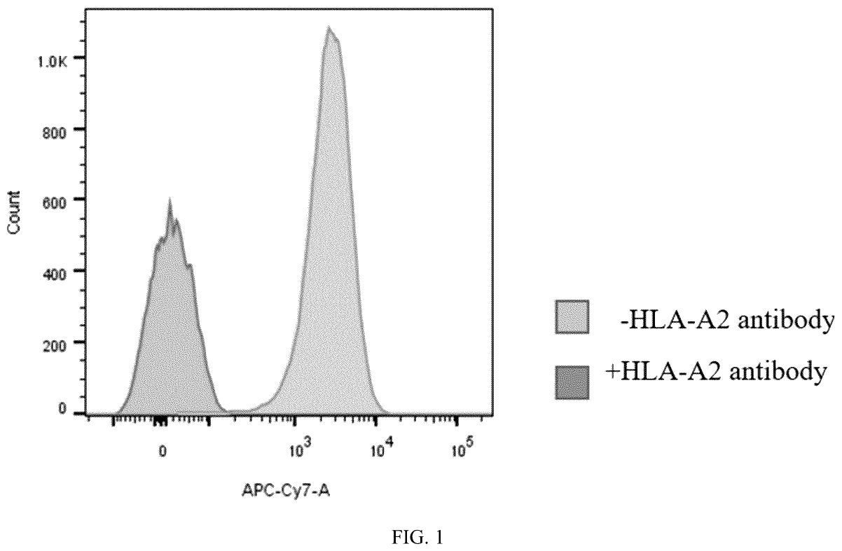

1. The HLA-A expression level on the surfaces of 293T cells was detected first. The culture condition of the 293T cells is a DMEM complete culture medium: DMEM+10% FBS+1% penicillin/streptomycin.
1) 2×10⁵ cells were taken from each of an experimental group and a control group. The cells in the experimental group was marked with HLA-A2 APC-Cy7 antibodies and incubated at 4° C. for 30 minutes.
2) 1 ml of PBE buffer solution was added to wash the antibodies, centrifugation was performed at 1500 rpm for 5 minutes to discard the supernatant, the cells was resuspended with 300 ul of PBE+2% FBS, and DAPI was added at a dilution of 1:1000 before on-machine detection.
3) The result of flow detection showed that the HLA-A protein was highly expressed on the surfaces of the flow 293T cells, which was significantly different from a negative group. The result indicates that the 293T cell can be used as a tool cell for HLA-A sgRNA library screening (the result is shown in FIG. 1).
2. Lentivirus was packaged by the HLA-A sgRNA library. The virus was packaged in a 10 cm culture dish. The virus packaging system is as follows (Table 1):

TABLE 1
10 cm culture dish

| | |
|---|---|
| PAX2 | 6 ug |
| VSVG | 3 ug |
| sgRNA library plasmid | 9 ug |
| PEI | (6 + 3 + 9) × 4 = 72 ug |

1) The DMEM complete culture medium was replaced with a 6 ml optim culture medium, and the 6 ml optim culture medium was put into an incubator.
2) A transfection system was prepared: a 1 ml optim culture medium was added into an EP tube, and PAX2, VSVG and sgRNA plasmid after swirling were added according to the above system.
3) Swirling and short centrifugal were performed.
4) PET was added according to the amount for blowing, beating and uniform mixing.
5) Standing was performed for 20 minutes.
6) The plasmid system was added into a dish and shaken well.
7) The 6 ml optim culture medium was replaced with the DMEM complete culture medium after 8 hours.
8) The virus supernatant was collected after 48 hours and then placed at 4° C.
3. The virus titer was measured.
1) 293T cells were paved on a 48-well plate one day in advance, with 6×10⁴ cells for each well and adopting a 300 ul DMEM complete culture medium.
2) the collected 2000 G virus supernatant was centrifuged for 5 minutes. The virus supernatant was filtered with a 0.45 um filter head.
3) A virus infection system was prepared according to Table 2, with 500 ug of infection system for each well, 6 virus gradients and duplicate for each gradient. In addition, 6 control wells without viruses were prepared.

TABLE 2

| Virus Volume | | Virus Infection System |
|---|---|---|
| 5 | ul | 10 ul virus supernatant + 990 ul DMEM complete |
| | | culture medium + 1 ul polybrene |
| 10 | ul | 20 ul virus supernatant + 480 ul DMEM complete |
| | | culture medium + 1 ul polybrene |
| 50 | ul | 100 ul virus supernatant + 400 ul DMEM complete |
| | | culture medium + 1 ul polybrene |
| 100 | ul | 200 ul virus supernatant + 800 ul DMEM complete |
| | | culture medium + 1 ul polybrene |
| 200 | ul | 400 ul virus supernatant + 600 ul DMEM complete |
| | | culture medium + 1 ul polybrene |
| 500 | ul | 1 ml virus supernatant + 1 ul polybrene |

4) The culture medium in a culture plate was sucked and added into the virus infection system.
5) 48 hours after infection, puromycin of 2 ug/ml was added into one duplicate cell in each virus gradient group and the control group for medicine screening, and the other well was used as control.
6) After 48-hour puromycin medicine screening, all cells in the control group should be observed to die. The number of living cells of the puromycin group and the puromycin-free group under each virus gradient was recorded.
7) The virus gradient was calculated according to a formula:

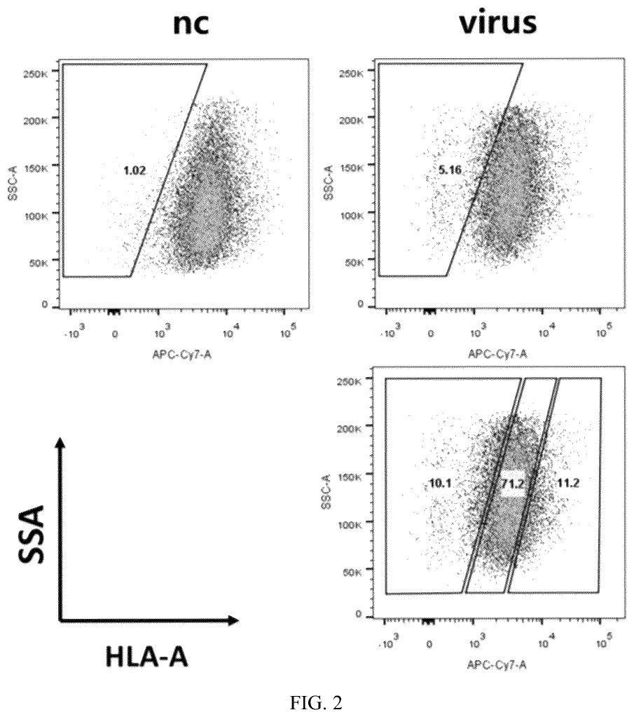

(60000*(the number of living cells in the puromycin group/the number of living cells in the puromycin-free group))/virus volume (ml)=virus titer TU/ml
4. A Cas9 stably transfected 293T cell line was infected with the virus supernatant.
1) Cell plating was performed in a 10 cm dish one day in advance, and the number of cells and the virus volume were calculated to make the virus infection rate less than 30%. Infection system: xml virus supernatant+(10-x)ml DMEM complete culture medium+10 ul polybrene. Three repeating groups and 1 virus-free control group were made.
2) 48 hours after virus infection, puromycin of 2 ug/ml was added for medicine screening. The knock-out situation of HLA-A was detected by a flow technology on the 17th day of medicine screening. It can be observed that the proportion of the cell population with HLA-A low expression in the virus group was increased compared with the control group. The cell population with HLA-A low expression of 10% and the cell population with high expression of 10% were sorted by the flow technology, and the unsorted cell population was kept (the result shown in FIG. 2).
5. The cell population with HLA-A low expression and the unsorted cell population genome were extracted, an sgRNA fragment was amplified by the PCR technology, and the amplified sgRNA fragment was subjected to NGS sequencing.
1) The cell population with HLA-A low expression and the unsorted cell population genome were extracted according to a TIANGEN DNA extraction kit, and the DNA concentration of each group was measured.
2) The sgRNA fragment was amplified by the PCR technology, with two steps of PCR:
PCR1 (345 bp)

### Primers:

CRISPR_PCR1_F:
5′-AGGGCCTATTTCCCATGATT
CRISPR_PCR1_R:
5′-CGGTGCCACTTTTTCAAGTT

### PCR1 System and Condition:

PCR1 (50 ul)

| PCR1 (50 ul) | | | programme | | |
|---|---|---|---|---|---|
| 5 × buffer | 10 | ul | 95° C. | 2 | min |
| dNTP | 0.5 | ul | 95° C. | 10 | sec |
| CRISPR_PCR_1F | 1 | ul | 57° C. | 20 | sec |
| CRISPR_PCR_1R | 1 | ul | 72° C. | 30 | sec |
| Polymerase(herculase) | 0.5 | ul | cycles | 23 for gDNA/20 | |
| | | | | for plasmids | |
| DMSO(final 3%) | 1.5 | ul | 72° C. | 5 | min |

| | | | | |
|---|---|---|---|---|
| gDNA/plasmids | 0.5 ug/5 ng | hold | 4° | C. |
| H₂O | add to 50 ul | | | |

PCR2 (265 bp)

### Primers:

B5xx_CRISPR_PCR_2F,
5′-:NNNNNNNNAGGCTGTTAGAGAGATAA
B7xx_CRISPR_PCR_2R,
5′-NNNNNNNNGCTGTTTCCAGCATAG

PCR2 System and Condition (Each Sample with 8 Reactions):

PCR2(50 ul)

| PCR2 (50 ul) | | programme | | |
|---|---|---|---|---|
| Polymerase(PrimeStar | 25 ul | 98° C. | 30 | sec |
| Max) | | | | |
| B5xx_CRISPR_{—} | 1 ul | 98° C. | 10 | sec |
| PCR_2F | | | | |
| B7xx_CRISPR_{—} | 1 ul | 60° C. | 15 | sec |
| PCR_2R | | | | |
| PCR1 | 1 ul | 72° C. | 20 | sec |

| | | | |
|---|---|---|---|
| H₂O | add to 50 ul | cycles | 18 for gDNA/12 |
| | | | for plasmids |

| | | |
|---|---|---|
| 72° C. | 5 | min |
| hold | 4° | C. |

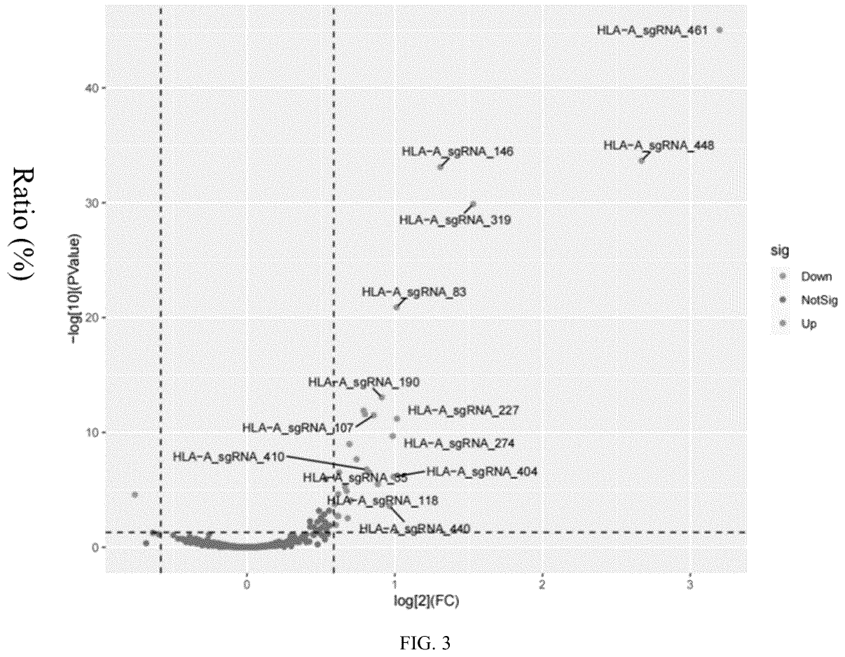

6. A PCR product was purified according to a ZYMO RESEARCH DNA purification kit method, and the DNA concentration was measured after purification. 500 ng of each sample was taken for second generation sequencing.
7. According to the sequencing result, the enrichment situation of the sgRNA of the cells in the HLA-A low expression group was subjected to bioinformatic analysis compared with that in the unsorted group. When the Fold change is greater than 1.5 and p is less than 0.05, 24 effective sgRNAs were screened out (the result shown in FIG. 3).
8. The database China MAP (http://www.mbiobank.com) was queried, where the database includes the entire HLA gene variation sites (SNP) of Chinese people and the frequency of the corresponding variation site. All SNP sites (95 mutation sites) of HLA-A provided by the database were compared with the 24 effective sgRNAs screened above to exclude 3 sgRNAs including the variation sites. The lowest SNP frequency provided by the database is less than 1/10000, so it is considered that the screened 21 sgRNAs (not including the variation sites) can cover more than 99% of Chinese population, and the sequence is shown in SEQ ID Nos. 1-21.

### Embodiment 2: The Knock-Out Effect of the Screened sgRNA was Inspected on the 293T Cell Line by a Transient Transfection Method

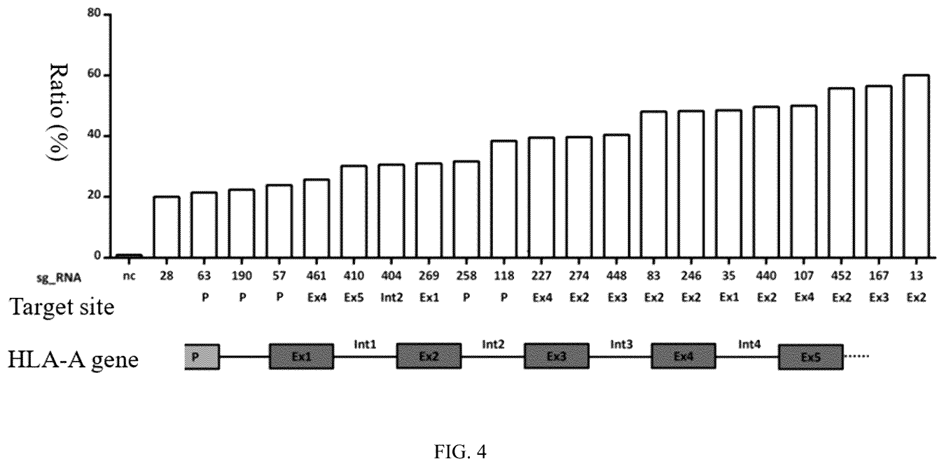

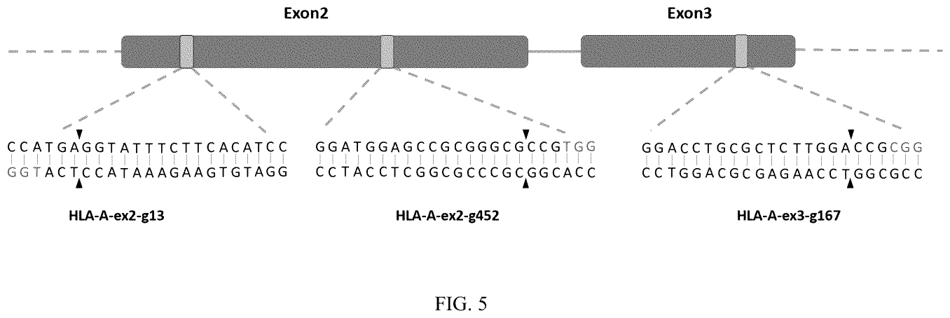

1) 293T cells were paved on a 24-well plate 24 hours in advance, with 2×10⁵ cells for each well.
2) The DMEM complete culture medium was replaced with a 0.4 ml DMEM culture medium (including 10% FBS and not including penicillin/streptomycin), and the Penicillin/streptomycin was put into an incubator.
3) A transfection system was prepared: a 50 ul optim culture medium was first added into a 1.5 ml EP tube, target plasmid of 1 ug/well (0.5 ug/well for each of the sg plasmid and the Cas9 plasmid, and the sg plasmid prepared by a conventional method) was added, and inverted mixing was performed without swirling.
4) Another 1.5 ml EP tube was taken, 50 ul optim culture medium was first added, PEI of 3 ug/well was added, and inverted mixing was performed without swirling.
5) The mixture was placed at room temperature for 5 minutes.
6) 3) and 4) were mixed into one tube, inverted mixing was performed without swirling, and standing was performed for 20 minutes.
7) The plasmid system was added into a dish and shaken well.
8) The 0.4 ml DMEM culture medium was replaced with the DMEM complete culture medium after 12-16 hours.
9) 48 hours after transfection, puromycin of 2 ug/ml was added for medicine screening.
10) 48 hours after puromycin medicine screening, the HLA-A knock-out effect of each sgRNA was detected.
11) The flow detection result shows that in 21 sgRNAs, the knock-out effects of HLA-A-ex2-g13 (SEQ ID No. 1), HLA-A-ex3-g167 (SEQ ID No. 2) and HLA-A-ex2-g452 (SEQ ID No. 3) are better, where the proportion of the HLA-A knock-out cells in the HLA-A-ex2-g13 can reach 60%. (The result is shown in FIG. 4).
12) The knock-out sites of HLA-A-ex2-g13 (SEQ ID No. 1), HLA-A-ex3-g167 (SEQ ID No. 2) and HLA-A-ex2-g452 (SEQ ID No. 3) on the human HLA-A gene are shown in FIG. 5.

### Embodiment 3: The Knock-Out Effect of the Screened sgRNA was Detected on Hematopoietic Cells Derived from Umbilical Cord Blood

1. CD34⁺ cells of umbilical cord blood were enriched, and the cells were put in a hematopoietic cell basic culture medium overnight. The hematopoietic cell basic culture medium is an SFEMII culture medium added with the following cell factors (Table 3):

TABLE 3

| Cell Factor | Use Concentration |
|---|---|
| hSCF | 100 ng/ml |
| hFlt3-L | 50 ng/ml |
| hTPO | 100 ng/ml |

2. Three sgRNAs with better knock-out effect on the 293T cells were selected: HLA-A-ex2-g13 (SEQ ID No. 1), HLA-A-ex3-g167 (SEQ ID No. 2) and HLA-A-ex2-g452 (SEQ ID No. 3), and the HLA-A protein on the surfaces of CD34⁺ cells were knocked out according to a Lonza P3 Primary Cell 4D-Nucleofector™ X Kit S kit electrotransfection method and through an electrotransfection RNP method.
1) CD34⁺ cells were counted, with 2×10⁵ cells for each sample.
2) An electrotransfection system was prepared: buffer was added in each tube first, and then 75 pmol of spCas9 protein and 150 pmol of sgRNA were added, totaling 5 ul. Reaction was performed at room temperature for 20-30 minutes.
3) Cells were resuspended with 15 ul of buffer, RNP was added (prepared into a 20 ul system), and the system was blown and beaten for more than 10 times. Liquid was added without adding bubbles.
4) Operation was performed according to the instructions of a 4D electrotransfection instrument.
5) After the electrotransfection, cell suspension in an electrotransfection group was transferred to a 1.5 ml centrifugal tube. 500 g was centrifuged for 5 minutes and the supernatant was discarded.
3. The cells were transferred to a 24-well plate for culture and observation. After 72 hours, the expression situations of HLA-A, HLA-ABC and HLA-DR on the surfaces of CD34⁺, CD34⁺CD90⁺ and CD34⁺CD90⁻ cells were respectively detected by a flow technology. The antibodies labeled by the flow technology are shown in Table 4.

TABLE 4

| Detection Protein | Fluorescein | Antibody (ul/sample) |
|---|---|---|
| CD34 | PE | 1 |
| CD90 | percp-cy5.5 | 1 |
| HLA-A2 | APC-cy7 | 0.5 |
| HLA-ABC | APC | 0.5 |
| HLA-DR | FITC | 0.5 |

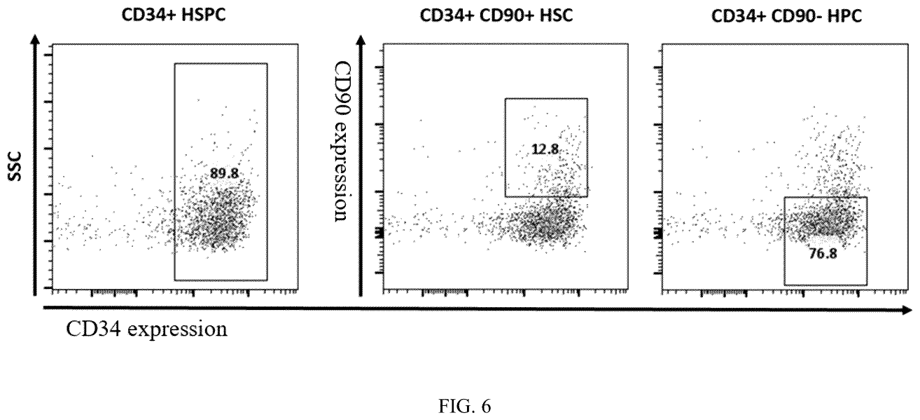

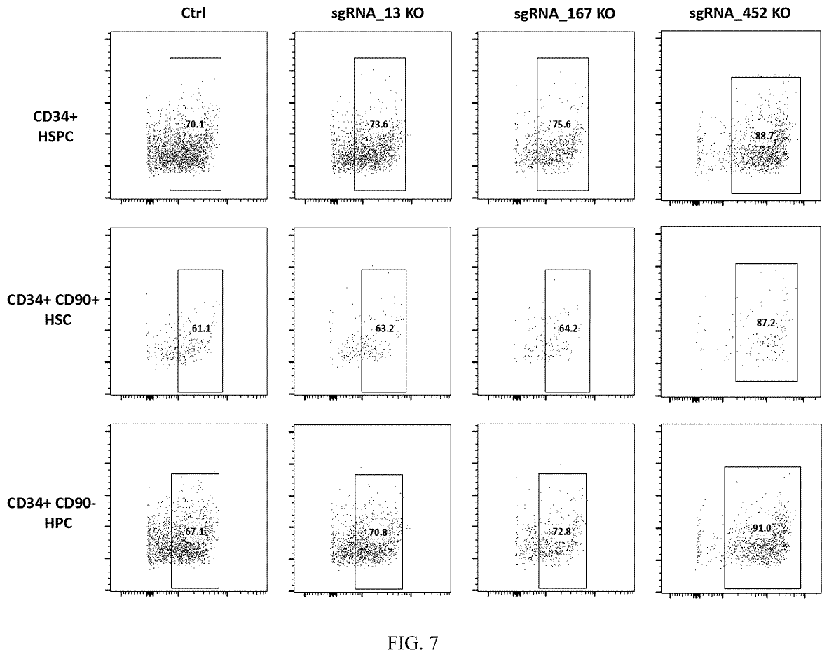

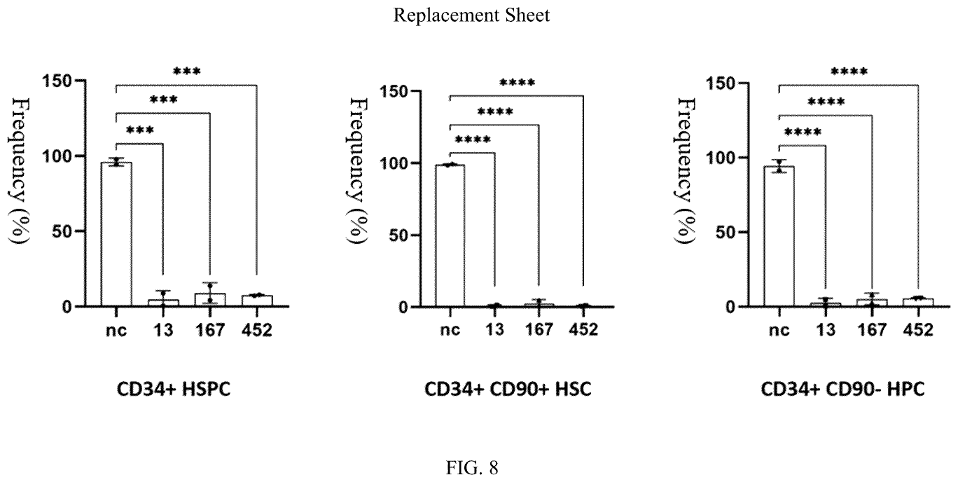

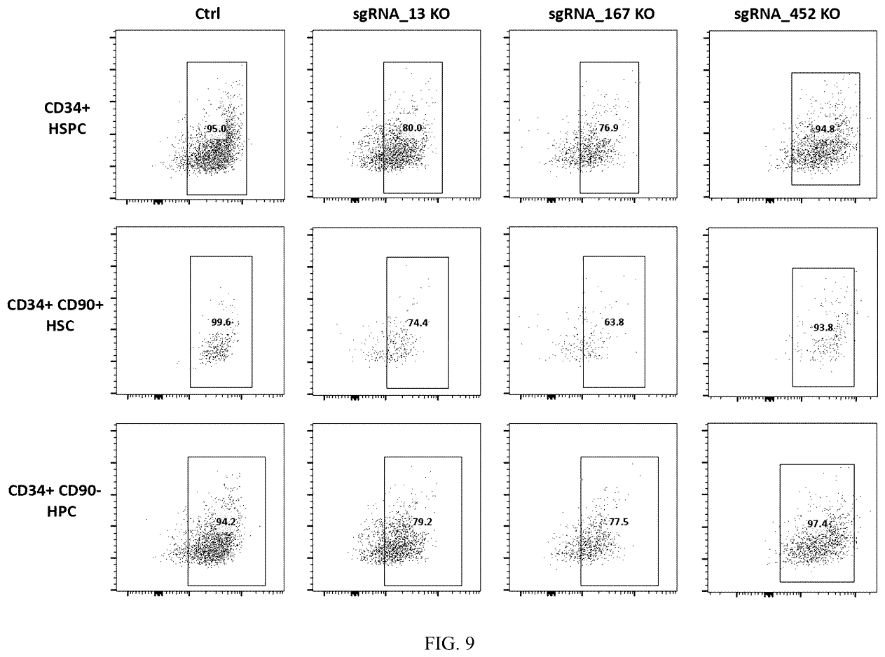

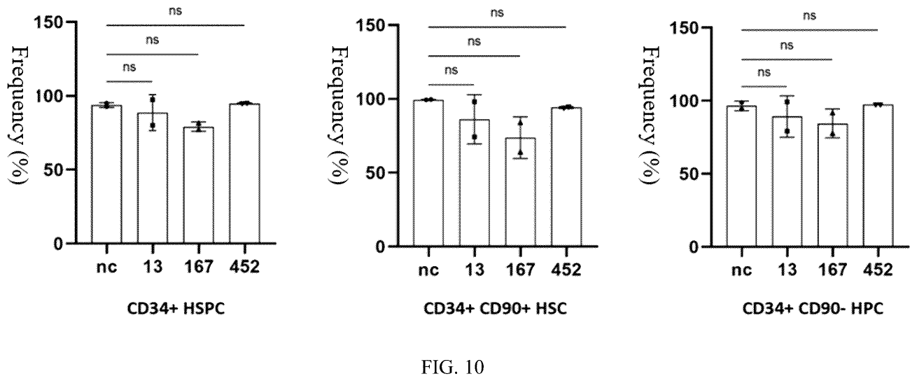

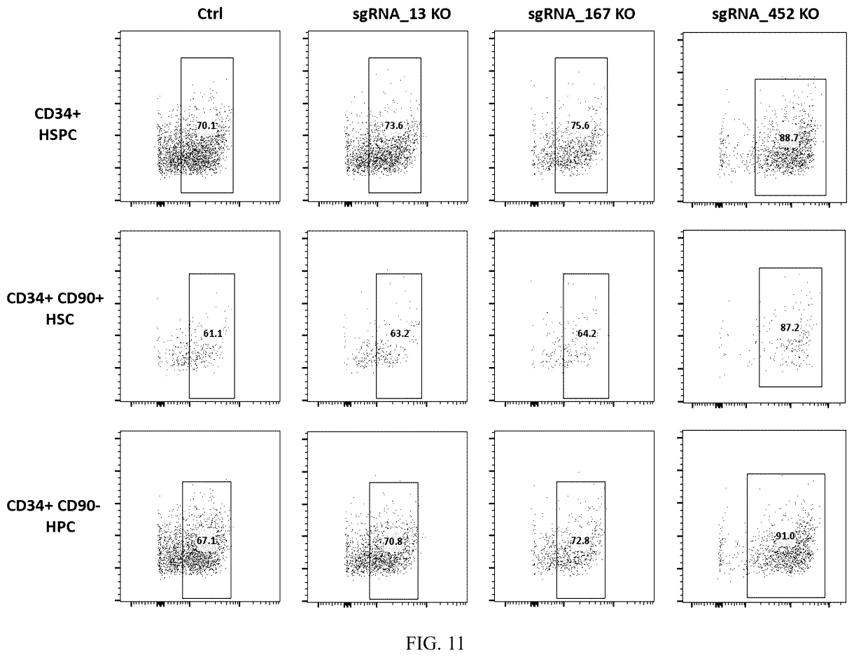

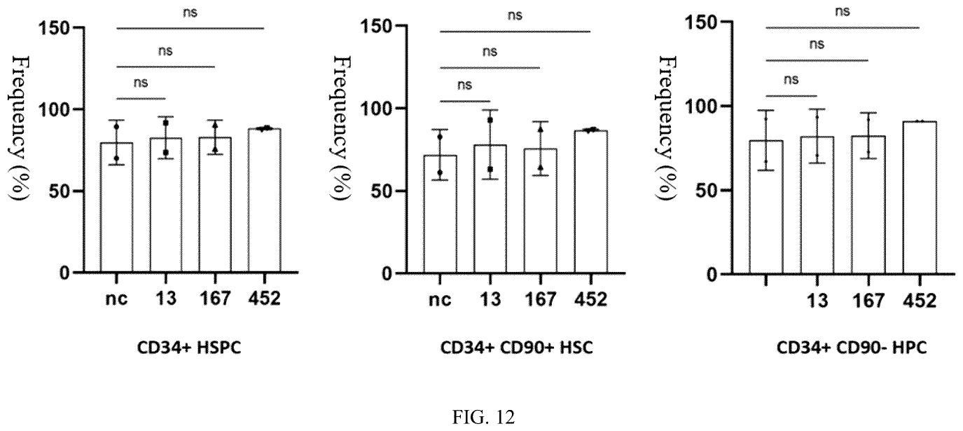

4. The flow results show that three sgRNAs can significantly knock out the HLA-A protein on the surfaces of CD34⁺, CD34⁺CD90⁺ and CD34⁺CD90⁻ cells, the knock-out proportion of the CD34⁺ cells by the three sgRNAs are 94% or above, and the HLA-A knock-out cell proportion on the CD34⁺CD90⁺ cells in the HLA-A-ex2-g13 group can reach 97% (FIG. 6 is a flow gating strategy, and the results are shown in FIG. 7 and FIG. 8). In addition, there is no significant change in the proportion of the HLA-ABC and HLA-DR positive populations on the CD34⁺, CD34⁺CD90⁺ and CD34⁺CD90⁻ cells in the three sgRNA groups. After statistical analysis, there is no significant influence (the results shown in FIG. 11 and FIG. 12) on the expression of HLA-B and HLA-C(the results shown in FIG. 9 and FIG. 10) and HLA-II molecules on three groups of cell HLA-I molecules after the HLA-A is knocked by the three sgRNAs, the sgRNA provided by the present disclosure has high targeting.

The above are preferred embodiments of the present invention, and it should be noted that, for those of ordinary skill in the art, several improvements and modifications may be made without departing from the principle of the present invention, and the improvements and modifications are also regarded to be within the protection scope of the present invention.

What is claimed is:

1. An sgRNA, wherein the nucleotide sequence of the sgRNA is shown in SEQ ID No. 1.

2. The sgRNA according to claim 1, wherein the nucleotide sequence is modified.

3. A composition of sgRNA, comprising sgRNAs of the nucleotide sequences shown in SEQ ID No. 1, SEQ ID No. 2 and SEQ ID No. 3.

4. The composition according to claim 3, wherein the nucleotide sequence is modified.

5. A recombinant vector, comprising the nucleotide sequence in the sgRNA according to claim 1.

6. A recombinant vector, comprising the nucleotide sequence in the composition according to claim 4.

7. The recombinant vector according to claim 5, further comprising a nucleotide sequence encoding Cas protein.

8. The recombinant vector according to claim 6, further comprising a nucleotide sequence encoding Cas protein.

9. A kit, comprising the sgRNA according to claim 1.

10. The kit according to claim 9, further comprising Cas protein or an expression vector of Cas protein.

11. A drug composition, comprising the sgRNA according to claim 1, and a pharmaceutically acceptable vector.

12. A method for improving the efficiency of knocking out or knocking down a human HLA-A gene in a cell, comprising:
step 1): transferring the sgRNA according to claim 1 into the cell;
step 2): transferring Cas protein or an expression vector of Cas protein into the cell; and
step 3) growing the cell, wherein
the method is for a non-therapeutic purpose.

13. The method according to claim 12, wherein the cell grows in an in vitro culture environment.


SEQUENCE LISTING (21 sequences)

SEQ ID NO: 1 - RNA, 20 nt, synthetic construct
  source 1..20: mol_type = other RNA
ggatgtgaagaaatacctca

SEQ ID NO: 2 - RNA, 20 nt, synthetic construct
  source 1..20: mol_type = other RNA
ggacctgcgctcttggaccg

SEQ ID NO: 3 - RNA, 20 nt, synthetic construct
  source 1..20: mol_type = other RNA
ggatggagccgcgggcgccg

SEQ ID NO: 4 - RNA, 20 nt, synthetic construct
  source 1..20: mol_type = other RNA
ggccgcctcccacttgcgct

SEQ ID NO: 5 - RNA, 20 nt, synthetic construct
  source 1..20: mol_type = other RNA
ctggttgtagtagccgcgca

SEQ ID NO: 6 - RNA, 20 nt, synthetic construct
  source 1..20: mol_type = other RNA
cgtgtcccggcccggccgcg

SEQ ID NO: 7 - RNA, 20 nt, synthetic construct
  source 1..20: mol_type = other RNA
cacagccgcccacttctgga

SEQ ID NO: 8 - RNA, 20 nt, synthetic construct
  source 1..20: mol_type = other RNA
attgctggcctggttctcct

SEQ ID NO: 9 - RNA, 20 nt, synthetic construct
  source 1..20: mol_type = other RNA
acccaatgggagtgagaact

SEQ ID NO: 10 - RNA, 20 nt, synthetic construct
  source 1..20: mol_type = other RNA
gtcctccccatcccgctgcc

SEQ ID NO: 11 - RNA, 20 nt, synthetic construct
  source 1..20: mol_type = other RNA
gagccagaggatggagccgc

SEQ ID NO: 12 - RNA, 20 nt, synthetic construct
  source 1..20: mol_type = other RNA
acccagttctcactcccatt

SEQ ID NO: 13 - RNA, 20 nt, synthetic construct
  source 1..20: mol_type = other RNA
gagagggagaaaagaaactg

SEQ ID NO: 14 - RNA, 20 nt, synthetic construct
  source 1..20: mol_type = other RNA
aggtcagtgtgatctccgca

SEQ ID NO: 15 - RNA, 20 nt, synthetic construct
  source 1..20: mol_type = other RNA
gaccccgcactcacccgccc

SEQ ID NO: 16 - RNA, 20 nt, synthetic construct
  source 1..20: mol_type = other RNA
ctggaaacccgacacccaat

SEQ ID NO: 17 - RNA, 20 nt, synthetic construct
  source 1..20: mol_type = other RNA
gagggttcggggcgccatga

SEQ ID NO: 18 - RNA, 20 nt, synthetic construct
  source 1..20: mol_type = other RNA
tctggaaacccgacacccaa

SEQ ID NO: 19 - RNA, 20 nt, synthetic construct
  source 1..20: mol_type = other RNA
gggggactgggctgaccgcg

SEQ ID NO: 20 - RNA, 20 nt, synthetic construct
  source 1..20: mol_type = other RNA
cggctccatcctctggctcg

SEQ ID NO: 21 - RNA, 20 nt, synthetic construct
  source 1..20: mol_type = other RNA
gggagaatctgagtcccggt